(19) United States
(12) Patent Application Publication
(10) Pub. No.: US 2011/0003012 A1
(43) Pub. Date: Jan. 6, 2011

(54) TREATING PATIENTS WITH SUBARACHNOID HEMORRHAGE

(75) Inventors: Jonathan S. Stamler, Chapel Hill, NC (US); David S. Warner, Durham, NC (US); James D. Reynolds, Durham, NC (US); Huaxin Sheng, Durham, NC (US)

Correspondence Address: BACON & THOMAS, PLLC, 625 SLATERS LANE, FOURTH FLOOR, ALEXANDRIA, VA, 22314-1176, US

(73) Assignee: Duke University, Durham, NC (US)

(21) Appl. No.: 12/677,336

(22) PCT Filed: Sep. 8, 2008
(86) PCT No.: PCT/US08/10453

§ 371 Date: Sep. 17, 2010

Related U.S. Application Data
(60) Provisional application No. 60/935,991, filed on Sep. 10, 2007.

(51) Int. Cl. A61K 33/26; A61K 31/04; A61K 33/00; A61K 31/198; A61P 7/04

(52) U.S. Cl. 424/646; 514/740; 424/718; 514/562

(57) ABSTRACT

A method for attenuating vasoconstriction in a patient with subarachnoid hemorrhage by administering to the patient a therapeutically effective amount of a compound which mediates an increase of bioactive nitric oxide in blood or tissue in the subarachnoid space to cause vasodilation in cerebral, carotid and basilar arteries after the administration of the compound, and wherein the administration of the compound does not reduce mean arterial blood pressure by more than 10%.

This application claims priority to U.S. Provisional Application No. 60/935,991, the entirety of which is incorporated herein by reference.

### TECHNICAL FIELD

This invention is directed to method for reducing the likelihood or severity of vasospasm, namely blood vessel constriction, in patients with subarachnoid hemorrhage; such vasospasm can cause secondary ischemia.

### BACKGROUND OF THE INVENTION

Subarachnoid hemorrhage (SAH) constitutes sudden bleeding (extravasation of blood) into the subarachnoid space of the central nervous system. SAH is classified as spontaneous or traumatic. Spontaneous SAH usually results from a ruptured intracranial aneurysm. Traumatic SAH usually results from a bicycle, motorcycle or automobile accident or accidental fall or a sports related cause.

Symptoms of subarachnoid hemorrhage include acute severe headache, vomiting, dizziness, loss of consciousness, coma, stiff neck, fever, aversion to light and neurologic deficits, e.g., partial paralysis, loss of vision, seizures and speech difficulties. Death occurs in about 35% of patients of a first aneurysmal hemorrhage. About 15% more die within a few weeks after a second rupture.

Diagnosis of subarachnoid hemorrhage can be made based on symptoms, computed tomography scan, cerebral angiography, magnetic resonance imaging and lumbar puncture and examination of cerebrospinal fluid (indicated by red blood cells in cerebrospinal fluid and/or yellowish tinge to cerebrospinal fluid (caused by blood breakdown products)).

A common complication is vasospasm, i.e., blood vessel constriction, resulting in secondary ischemia and neurologic deficits. This may last for days to weeks. About one-third of spontaneous subarachnoid hemorrhage events are followed by vasospasm. Vasospasm occurs in 30-60% of traumatic cases.

Current treatments for prevention of vasospasm include the calcium channel blocker nimodipine and hypertensive/hemodilution therapy (artificial elevation of blood pressure to promote flow of blood). These treatments are generally unsuccessful.

It is posited that vasodilation will prevent or attenuate the occurrence of vasospasm, and that this may be effected by increasing the presence of the vasodilator nitric oxide.

One attempt to prove this theory, i.e. nitric oxide increase will prevent vasospasm, involved using a mouse model transgenically modified to express increased amount of extracellular superoxide dismutase. The concept was that the superoxide dismutase would cause a decrease in the amount of superoxide available to react with nitric oxide thereby better preserving the availability of endogenous nitric oxide to act as vasodilator to reduce occurrence of cerebral vasospasm. The superoxide dismutase overexpression decreased vasospasm, but not enough to cause a change in neurologic deficits caused by vasospasm. See McGirt, M. J., et al., Stroke 33, September 2002, 2317-2323.

Another attempt to prove this theory involved administration of a nitric oxide donor. Sodium nitroprusside, a non-selective vasodilator, administered intravenously has not been a suitable treatment to reduce occurrence and severity of vasospasm, because when used alone, the dose required to cause cerebral vasodilation also causes major systemic hypertension.

In another case, mice with induced subarachnoid hemorrhage were treated with simvastatin (a drug used to treat hypercholesterolemia in humans). Simvastatin is known to increase endothelial nitric oxide synthase in humans, thereby increasing the amount of nitric oxide produced. This treatment worked to cause reduction in occurrence of vasospasm in the mice. See McGirt, M. J., et al., Stroke 33, 2950-2956 (December 2002). A trial on a small group of humans with subarachnoid hemorrhage produced the same results. The simvastatin acted but slowly, requiring more than one day to achieve the desired effect. Moreover, simvastatin has also been found to have relatively weak vasodilatory effects and the dose is limited by muscle and liver toxicity.

A similar result was obtained by others with pravastatin. A 1500 patient study is underway in the UK to address the effectiveness of statin therapy to reduce the occurrence of vasospasm after subarachnoid hemorrhage but no results have been announced as of yet.

In another case, monkeys that had an autologous blood clot placed around the right middle cerebral artery were treated with low dose sodium nitrite intravenous solution over 24 hours along with a sodium nitrate bolus, daily, or higher dose sodium nitrite solution infused over 24 hours with no bolus, daily, or control saline solution infusion. The lower dose plus bolus resulted in less vasospasm than higher dose treatments but transient (after bolus) blood pressure reduction by more than 15% occurred. The higher dose infusion with no bolus resulted in more vasospasm than the lower dose plus bolus. Both reduced vasospasm compared to control. See Pluta, R. M., et al, JAMA 293(12), 1477-1484 (Mar. 23/30, 2005). However, both treatments were considered defective because transient blood pressure reduction by more than 15% can aggravate stroke. The higher dose treatment was deficient in reducing vasospasm only to 20% in two of three cases and created steal of blood flow from other parts of the brain. See FIG. 3 of Pluta et al. The potency of sodium nitrite is also a drawback. Even a “low” dose of nitrite requires the administration of amounts that are considered exceptionally high by pharmacological standards (order of magnitude higher than nitroprusside for example), which reflects the impotence of the drug. When drugs need to be administered at such high doses, it suggests that the drugs function on the basis of off target effects. A higher potency agent that does not drop blood pressure or create steal from other parts of the brain would be desirable.

### SUMMARY OF THE INVENTION

It is an object of this invention to administer a compound or combination of compounds, which cause an increase of bioactive nitric oxide in blood and tissue in the subarachnoid space to cause lasting and potent vasodilation in cerebral, carotid and basilar arteries after administering the compound or compounds without reducing mean arterial blood pressure by more than 10%. A feature that distinguishes the present invention from other procedures is the ability of the present invention to offer the onset of treatment effect immediately after diagnosis. Indeed, while the invention can “treat” vasospasm, one of the major goals of the invention to reduce the likelihood or severity of vasospasm and subsequent ischemic results. In one embodiment, the invention is directed to a method for attenuating or preventing pathological cerebral vasoconstriction in a patient with subarachnoid hemorrhage by administering to the patient a therapeutically effective amount of a compound which mediates an increase of bioactive nitric oxide in blood or tissue in the subarachnoid space to cause vasodilation in cerebral, carotid and basilar arteries after the administration of the compound, and wherein the administration of the compound does not reduce mean arterial blood pressure by more than 10%.

In another embodiment, the invention is directed to a method for reducing the likelihood and/or severity of vasospasm by administering to the patient a therapeutically effective amount of a compound which mediates an increase of bioactive nitric oxide in blood or tissue in the subarachnoid space to cause vasodilation in cerebral, carotid and basilar arteries after administration of the compound, and wherein the administration of the compound does not reduce mean arterial blood pressure by more than 10%.

In another embodiment, the invention is directed to a method for treating subarachnoid hemorrhage in a patient having had such, the method comprising the step of delivering into the lungs of the patient as a gas a vasospasm preventing or attenuating amount of ethyl nitrite within seven to 10 days of the occurrence of the subarachoid hemorrhage. Delivery into the lungs provides more rapid and direct access to the central nervous system than intravenous administration of sodium nitrite, and does not cause drop in mean arterial blood pressure by more than 10%, and does not worsen oxygenation contrary to the case with systemic vasodilation where this is a concern. Also, the potency of organic nitrites such as ethyl nitrite (ENO) is orders of magnitude greater than that of inorganic nitrite.

In another embodiment of the invention, the invention is directed to a method for treating subarachnoid hemorrhage in a patient having had such comprising, within three days of the diagnosis of the occurrence of subarachnoid hemorrhage as determined, for example, by computed tomography scan, or cerebral angiography, administering to the patient a vasospasm preventing or attenuating amount of an organic nitrite with or without an inorganic nitrite. This method offers several advantages relative to treatments only with intravenous sodium nitrite in that organic nitrite has been found to be more potent than inorganic nitrite and inorganic nitrate. As a result, the dosages are much smaller and the chances for toxicity (hypotension, methemoglobinemia, mutagenesis, tissue injury, respiratory block in mitochondria, hypoxemia) are far smaller. Moreover, inorganic nitrites when combined with organic nitrites exhibit unexpected and synergistic results, and can be administered in lower dosages in combination with organic nitrite than without organic nitrite

In yet another embodiment of the invention, the invention is directed to a method for treating subarachnoid hemorrhage in a patient having had such comprising, within three days of diagnosis of the occurrence of the subarachnoid hemorrhage, as determined, for example, by computed tomography or cerebral angiography, administering to the patient a vasospasm preventing or attenuating amount of a nitrosylating agent supplemented with an inorganic nitrite or organic nitrite, the amount being insufficient to reduce mean arterial blood pressure by more than 10%. The advantages for nitrosylating agent are the same as for organic nitrite described above. The drugs of the combinations here have different mechanisms of action and the combination accommodates for deficiencies in the treatment with less potent inorganic nitrite.

### BRIEF DESCRIPTION OF THE DRAWINGS

FIG. 1 is a plot of right internal carotid artery diameter in mice subjected to sham (Sham) surgery or subarachnoid hemorrhage (SAH). Mice were treated with 20 ppm ENO by inhalation for 3 days after sham surgery or SAH. Open circles indicate values for individual mice. Horizontal bars indicate group mean values.

FIG. 2 is a plot of right anterior cerebral artery diameter in mice subjected to sham (Sham) surgery or SAH. Mice were treated with 20 ppm ethyl nitrite (ENO) by inhalation for 3 days after sham surgery or SAH. Open circles indicate values for individual mice. Horizontal bars indicate group mean values.

FIG. 3 plot of right middle cerebral artery diameter in mice subjected to sham (Sham) surgery or SAH. Mice were treated with 20 ppm ENO by inhalation for 3 days after sham surgery or SAH. Open circles indicate values for individual mice. Horizontal bars indicate group mean values.

FIG. 4 is a plot of the latency to fall from a rotating rod 3 days after either sham surgery or SAH as a function of baseline normal values. Mice were treated with 20 ppm ENO by inhalation for 3 days after sham surgery or SAH. Open circles indicate values for individual mice. Horizontal bars indicate group mean values.

FIG. 5 demonstrates the specificity of S-nitrosylation of hemoglobin. FIG. 5A shows red blood cell lysate nitrosothiol (SNO) is increased approximately 4-fold by 3 days exposure to 20 ppm ENO. FIG. 5B shows this accounts for much of the increase in total red blood cell NO caused by ENO. FIG. 5C shows the change in serum total NO is negligible after 3 days exposure to 20 ppm ENO.

FIG. 6 shows the results of a study in which mice were subjected to subarachnoid hemorrhage and treated with air or ethyl nitrite.

### DETAILED DESCRIPTION

The term “attenuate” means to reduce the severity of, or to inhibit a recited condition or phenomenon (e.g., vasoconstriction). The term encompasses treating a patient having or at risk of developing the recited condition or phenomenon (i.e., vasoconstriction).

The phrase “pathological cerebral vasoconstriction” refers to clinically relevant or symptom inducing form of vasoconstriction.

As noted above, it is an object of this invention to administer a compound or combination of compounds which cause an increase of nitric oxide activity in blood and tissue in the subarachnoid space to cause lasting vasodilation in cerebral, carotid and basilar arteries after administering the compound or compounds without reducing mean arterial blood pressure by more than 10%.

The compound or combination of compounds can be administered to attenuate, treat, or prevent pathological cerebral vasoconstriction in a patient with subarachnoid hemorrhage. Accordingly, the compound or compounds can be administered to reduce the likelihood or severity of vasospasm in a patient.

In other words, the compound or compounds can be administered before or during the onset of vasospasm. This is believed to be possible because the compound or compounds can increase blood flow without decreasing blood pressure by more than 10%, are selective for ischaemic tissue, and reduce the likelihood or severity of vasospasm so as to decrease the chances of certain kinds of stroke from occurring.

The compound or combination of compounds that are administered preferably cause an increase of nitric oxide in blood and tissue in the subarachnoid space to cause lasting vasodilation in cerebral, carotid and basilar arteries one hour after being administered.

The compound or combination of compounds can be or include organic nitrites. The organic nitrites are optionally supplemented with inorganic nitrites. Additionally, the compound may be a nitrosylating agent supplemented with an inorganic nitrite and/or an organic nitrite.

The compound or combinations of compounds are administered within 0 to 10 days, and preferably within 3 to 7 days after the occurrence of the subarachoid hemorrhage, as determined by diagnosis of when the subarachnoid hemorrhage occurred. The administration of the compound or combination of compounds within 0 days indicates that the compound or combination of compounds can be administered promptly after the diagnosis has been made that a subarachnoid hemorrhage occurred. For example, the compound or combination of compounds can be administered within 1 minute to up to 10 days, preferably within 1 minute up to 2 days, more preferably within 1 hour to 10 days, and even more preferably 3 hours to 7 days after the occurrence of the subarachoid hemorrhage, as determined by diagnosis of when the subarachnoid hemorrhage occurred.

The compounds or combination of compounds are administered in dosages and routes as discussed below.

Time periods for administration of the compound or combination of compounds ranges from 1 minute to 2 days, 1 minute to up to 14 days, 1 minute to 2 days, 3 days to 10 days, and/or 3 to 7 days. The administration of the compound or combination of compounds should be carried out until the risk of vasospasm is no longer present, as determined by the attending physician.

We turn now to a preferred embodiment, wherein an ethyl nitrite is administered as a preferred organic nitrite.

Ethyl nitrite is commercially available diluted in ethanol. It is readily delivered to the patient in gaseous form by bubbling N₂ or O₂ through a Milligan gas diffuser containing ethyl nitrite diluted in ethanol (e.g., from 0.00125 to 0.5% ethyl nitrite in ethanol (v/v), preferably from 0.0025 to 0.125% ethyl nitrite in ethanol (v/v)), e.g., at a flow rate of 0.5 liters/min to 1.5 liters/min, preferably 0.75 liters/min to 1.25 liters/min, to produce N₂ or O₂ containing ethyl nitrite and introducing this into the ventilation system by mixing the output from the ventilator at a total of 1 to 10 liters/min, preferably 3 to 7 liters/min with the N₂ or O₂ containing ethyl nitrite, for example, to produce a concentration of 1 to 100 ppm ethyl nitrite in the resulting gas, and delivering this to the patient at a rate and pressure to maintain satisfactory Pa_{O2} and Pa_{CO2}. The concentration of ethyl nitrite gas administered is proportional to the flow rate of N₂ or O₂ and the concentration of ethyl nitrite liquid in ethanol. The flow rate into the patient ranges from 1 to 10 liters per minute, preferably 3 to 7 liters per minute.

Administration can also be carried out using a face mask.

Time periods for administration of organic nitrites including ethyl nitrite range from 1 minute to up to 14 days, for example 1 minute to 2 days, 3 to 7 days, and/or 3 days to 10 days. Administration is carried out until risk of vasospasm is no longer present, e.g., as determined by the attending physician.

We turn now to another preferred embodiment that involves administration of organic nitrite with or without administration of inorganic nitrite

The organic nitrite can be, for example, methyl nitrite, ethyl nitrite, tert-butyl nitrite or isoamyl nitrite. Organic nitrites can be prepared as described in Landscheidt U.S. Pat. No. 5,412,147.

We turn now to the administration of these compounds. Those that are normally gases are readily administered diluted in nitrogen or other inert gas and can be administered in admixture with oxygen. Those that are not normally gases can be converted to gas for administration and are administered diluted as in the case of the NO-containing compounds that are normally gases. The compounds should not have a boiling point such that the temperature required to maintain them as gases in diluted form would harm the lungs and preferably would not condense in the lungs. Dilution, for example, to a concentration of 1 to 100 ppm, preferably 25-75 ppm, and more preferably 40-60 ppm, is typically appropriate. The diluted gas is readily delivered into the lungs, using a ventilator which is a conventional device for administering gases into the lungs of a patient. A tube attached to the device passes the gas into the lungs at a rate and pressure consistent with maintaining a Pa_{O2} greater than or equal to 90 mm Hg.

Time periods for administration of organic nitrites range from 1 minute to up to 14 days, preferably range from 3 days to 10 days, more preferably 3 to 7 days, and even more preferably 1 minute to 2 days. Again, administration is carried out until risk of vasospasm is no longer present, e.g., as determined by the attending physician.

Administration can also be carried out using a face mask.

Organic nitrites that are not normally gases can also be administered, dissolved in ethanol and other solvents administered intravenously at a dosage of 1 nM to 10 micromolar final concentration, for example 3 nM to 7 micromolar final concentration estimated in blood.

The inorganic nitrite can be, for example, sodium or potassium nitrite and is administered dissolved, for example, in water for injection or saline intravenously at a dosage of 10 nM to 50 micromolar, for example 15 nM to 35 micromolar final plasma concentration.

We turn now to turn another embodiment of the invention, i.e., the embodiment of the invention involving administering nitrosylating agents supplemented with inorganic nitrite and/or organic nitrite.

The nitrosylating agent can be, for example, an O-nitroso compound, e.g. those mentioned in U.S. Pat. No. 6,472,390, also administered IV, an N-nitroso compound, e.g., DETANO, i.e., diethylene triamine NONOate, an S-nitroso compound, e.g. S-nitrosoglutathione, S-nitrosopenicillamine, and those listed in U.S. Pat. No. 6,472,390 and U.S. Pat. No. 6,314,956, an iron nitroso compound, e.g., sodium nitroprusside, and C-nitroso compounds, e.g., those mentioned in U.S. Pat. No. 7,049,308.

These can be administered intravenously in solvent at a dosage of 1-100 nmol/kg, for example at a dosage of 25-75 nmol/kg, and more preferably at a dosage of 40-60 nmol/kg (assessing that mean arterial blood pressure does not drop by more than 15%).

The inorganic nitrites are, for example, those listed in the embodiment involving administration of organic nitrite and administered in doses and routes of administration as described in that embodiment.

The organic nitrite is administered in doses and methods of administration as described above.

In all the embodiments mean arterial blood pressure is not reduced by more than 10%. All the embodiments provide the advantages of 1) increasing blood flow without significantly changing blood pressure; 2) selectively increasing blood flow to ischemic tissue; and 3) lessening the likelihood of stroke.

The invention is further supported by the following Background Examples and illustrated by the following Working Examples.

### Background Example 1

The following Background Example provides a set of method and procedures relevant to study the effects of nitric oxide. C57BL/6J mice (Jackson Laboratory, Bar Harbor, Me., USA) were fasted from food for 12 h to control their plasma glucose concentration. Anesthesia was induced in a chamber with 5% halothane in 50% O2/balance N2. The trachea was intubated and the lungs were mechanically ventilated. Pericranial temperature was maintained at 37.0°±0.5° C. using a heat lamp and a pericranial needle thermistor. Anesthesia was maintained with 0.6%-1.0% halothane in 50% O2/balance N2. By surgical incision, a catheter was placed in the left femoral artery. Mean arterial blood pressure (MAP) was maintained between 60-80 mmHg throughout surgery by adjusting the inspired halothane concentration. Arterial pH, PaCO2, and PaO2 were measured immediately before SAH.

SAH was generated in each subject as follows. The right common carotid artery was exposed by a midline incision of the neck and the external carotid artery (ECA) was isolated and ligated. A blunted 5-0 monofilament nylon suture was introduced into the ECA and advanced into the internal carotid artery (ICA). The suture was advanced distal to the right anterior cerebral artery (ACA)-middle cerebral artery (MCA) bifurcation, where resistance was encountered, and then advanced 3.0 mm further to perforate the right ACA. The suture was immediately withdrawn, allowing reperfusion and SAH. In sham mice, the suture was advanced only until the point of resistance thereby avoiding arterial perforation. After removal of the filament, the skin was closed with suture in both groups. Halothane anesthesia was discontinued. Upon recovery of spontaneous ventilation, the trachea was extubated. Mice were continuously observed until recovery of the righting reflex and were then returned to their cages. Subcutaneous injections of 10% dextrose in 0.9% NaCl in water (0.5 ml) were given twice per day to all of the mice to standardize hydration.

A neurobehavioral examination (cumulative scoring scale 5-27) was performed at 72 h after SAH or sham surgery. The exam included a motor score (0-12) derived from observed spontaneous activity, symmetry of limb movements, climbing, balance and coordination and a sensory score (5-15) derived from body proprioception, vibrissae, visual, olfactory, and tactile responses, Sensory tests examined functions contralateral to SAH or sham surgery are shown in Table 1. A cumulative score of 27 indicates no neurologic deficit. A score of 5 indicates severe neurologic deficit.

TABLE 1
Neurological examination variables

| | Motor Score | | | |
|---|---|---|---|---|
| | 0 | 1 | 2 | 3 |
| Activity | No movement | Moves, no | 1-2 walls | 3-4 walls |
| (5 min in open field) | | walls Approached | approached | approached |
| Limb symmetry | Left forelimb | Minimal | Abnormal | Symmetrical |
| (suspend by tail) | no movement | movement | forelimb walk | extension |
| Climbing | Fails to hold | Holds <4 sec | Holds, no | Displaces |
| (on inverted metal mesh) | | | displacement | across mesh |
| Balance | Falls <2 sec | Falls >2 sec | Holds, no | Displaces |
| (on 2 cm diameter rod) | | | displacement | across rod |

| | Sensory score | | |
|---|---|---|---|
| | 1 | 2 | 3 |
| Proprioception | No reaction | Asymmetrical | Symmetrical |
| (cotton tip to bilateral | head turning | head turning | |
| neck) | | | |
| Vibrassae | No reaction | Asymmetrical | Symmetrical |
| (cotton tip to vibrissae) | | head turning | head turning |
| Visual | No reaction | Unilateral blink | Bilateral blink |
| (tip toward each eye) | | | |
| Olfactory | No sniffing | Brief sniff | Sniff >2 sec |
| (lemon juice on tip) | | | |
| Tactile | No reaction | Delayed | Immediate |
| (needle stick to palm) | | withdrawal | withdrawal |

In later studies, the rotarod was used instead of the neurologic scoring system because the rotarod has been found to be highly sensitive in identifying neurologic deficits and rotarod performance also correlates closely with cumulative neurologic score. Rotarod testing was performed by placing the animal on a rotating cylinder in a quiet room with constant illumination. The cylinder rotation rate is linearly increased over a 5 min interval. The interval that the animal is able to ambulate on the rod without falling is automatically recorded. Mice are subjected to this test in a series of 3 trials, with the performance of each series being averaged.

Mice underwent cerebral intravascular perfusion 72 h after surgery when vasospasm has been reported to peak in a mouse. Gelatin powder (7 g) was dissolved in 100 ml 0.9% NaCl and mixed with 100 ml India ink (3085-4 Ultradraw, Koh-1-Noor, Inc., Bloomsbury, N.J., USA). The solution was maintained at 50° C. and homogenized with a sonic homogenizer (Model G112SP1T, Laboratory Supplies Co. Inc., Hicksville, N.Y., USA). The mice were anesthetized with halothane. The tracheas were intubated and the lungs mechanically ventilated to maintain normocapnia. The chest was opened and the aorta was cannulated through the left ventricle with a blunted 23 gauge needle. Flexible plastic tubing (Tygon™, 3.2 mm internal diameter, VWR Scientific, West Chester, Pa., USA) was connected to a mercury manometer and a 0.062 mm diameter silicone tube attached to the 23 gauge needle to deliver perfusion solutions by manual pulsatile syringe pressure. All perfusates were filtered using a 0.2 mm pore size syringe filter. A 30 ml syringe was connected to this proximal closed system to deliver the perfusates. An incision was made in the right atrium to allow drainage of perfusion solutions. Normal saline (20 ml) was infused first followed by 15 min of 10% formalin and then 10 min of India ink/gelatin solution (cooled to 37° C.). Perfusion pressures were controlled using the manometer. The mouse was then refrigerated for 24 h to allow gelatin solidification. The brains were harvested and stored in 10% neutral buffer formalin.

Blood vessels were imaged using a video linked dissecting microscope controlled by an image analyzer. The image of each section was stored as a 1280×960 matrix of calibrated pixel units and displayed on a video screen. Two regions of the ipsilateral MCA were analyzed; a 1.0 mm segment proximal to the ACA-MCA bifurcation and a 1.0 mm segment distal to the bifurcation. The ipsilateral ICA and ACA were divided into proximal and distal 0.8 mm segments. The smallest lumen diameter within each vascular segment was measured from the digitized images by an observer blinded to the experimental group.

Brains were analyzed under light microscopy (×6 magnification) to determine the magnitude of SAH by an observer blinded to experimental group. Hemorrhage size was graded by two characteristics: area of hemorrhage distribution and density of clot formation. Hemorrhage area was scored as: 1=SAH extends anteriorly <1.0 mm from MCA-ACA bifurcation; 2=SAH extends >1.0 mm anteriorly from bifurcation; 3=SAH extends >1.0 mm anteriorly from bifurcation with posterior extension across the ICA. Hemorrhage density was scored as: I=underlying brain parenchyma visualized through clot; 2=underlying brain parenchyma not visualized through clot. Hemorrhage grade (2-5) was determined by the sum of the size and density scores. Absence of hemorrhage was scored as 0.

### Experiment A

To determine the effect of perfusion pressure on India ink/gelatin cast vascular diameters, 37 C57BL/6J mice, 8-10 weeks old, were randomized to SAH or sham surgery. Seventy-two hours after surgery, mice underwent India ink/gelatin casting at one of three perfusion pressures: 40-60 mmHg (seven sham, six SAH); 60-80 mmHg (seven sham, six SAH); or 100-120 mmHg (six sham, five SAH). All artery segments ipsilateral to surgery were measured as described. An additional four mice underwent perfusion without microfiltration at a perfusion pressure of 60-80 mmHg.

### Experiment B

To confirm the findings of Experiment A and to examine the relationship of vessel diameter, SAH grade, and neurological deficits, a second set of mice underwent SAH (n=8) or sham surgery (n=7). Seventy-two hours after surgery, mice underwent neurological examination as described above. Mice were then anesthetized with halothane and subjected to India ink/gelatin perfusion fixation at 60-80 mmHg with microfiltration of all perfusates. SAH was graded prior to vascular measurement. Proximal MCA diameter was measured and compared between groups. In order to attribute differences in artery diameters to local vasospasm and not variations in gelatin-ink perfusion, the basilar artery diameter was also measured in all mice.

Two-way analysis of variance (group versus perfusion pressure) was used to compare vessel diameters. When indicated by a significant F ratio, intergroup comparisons were made with Scheffe's test. Neurological scores were compared by the Mann-Whitney test. Correlations between neurological score, MCA diameter, and SAH grade were analyzed using Spearman rank correlation coefficient. Parametric values are given as mean=standard deviation. Nonparametric values are given as median (interquartile range). p<0.05 was considered statistically significant.

### Results of Experiments A and B

### Experiment A

Body weight prior to surgery was similar between groups (sham=33=4 g, SAH=33=3 g), but at three days after surgery, body weight was reduced in the SAH group (sham=32+7 g; SAH=29+3 g, p<0.05). Values for PaCO2 (sham=34±3 mmHg; SAH=33+3 mmHg), PaO2 (sham=168+21 mmHg; SAH=138+31 mmHg) and arterial pH (sham=7.24+0.01; SAH=7.27+0.05) were similar between groups. A decrease in vascular diameter was observed after SAH in the proximal and distal MCA and distal ICA segments at a pressure of 60-80 mmHg. At 40-60 mmHg, a decrease in diameter after SAH was observed in the distal ICA only (Table 2). No difference between sham and SAH groups was observed in any segment at 100-120 mmHg. A main effect (p<0.05) for increasing diameter as a function of increasing perfusion pressure was present in both sham and SAH groups in most vascular segments. This was most evident between the perfusion pressure ranges of 60-80 and 100-120 mmHg (Table 2). Without gelatin-ink microfiltration, all four mice demonstrated embolization artifacts preventing adequate vascular measurement.

TABLE 2
Arterial diameter (um) as a function of perfusate infusion pressure

| | Perfusion pressure (mmHg) | | |
|---|---|---|---|
| | 40-60 | 60-80 | 100-120 |
| Proximal MCA | | | |
| Sham | 92 ± 33 | 89 ± 18 | 139 ± 23 |
| SAH | 74 ± 59 | 50 ± 31* | 91 ± 43 |
| Distal MCA | | | |
| Sham | 93 ± 34 | 88 ± 21 | 122 ± 11 |
| SAH | 66 ± 52 | 48 ± 27* | 100 ± 60 |
| Proximal ACA | | | |
| Sham | 113 ± 38 | 115 ± 24 | 150 ± 14 |
| SAH | 80 ± 70 | 77 ± 39 | 133 ± 55 |
| Distal ACA | | | |
| Sham | 102 ± 31 | 103 ± 22 | 150 ± 14 |
| SAH | 79 ± 64 | 78 ± 42 | 118 ± 70 |
| Proximal ICA | | | |
| Sham | 147 ± 58 | 131 ± 34 | 187 ± 6 |
| SAH | 64 ± 73 | 71 ± 52 | 141 ± 60 |
| Distal ICA | | | |
| Sham | 132 ± 47 | 136 ± 30 | 170 ± 16 |
| SAH | 59 ± 60* | 74 ± 60* | 200 ± 18 |

SAH, subarachnoid score, MCA diameter, and SAH grade were analyzed using Spearman rank correlation coefficient. Parametric values are given as mean = standard deviation. Nonparametric values are given as median (interquartile range). p < 0.05 was considered statistically significant.

### Experiment B

Physiologic values were similar to those reported for Experiment 1. SAH caused a 57% reduction in proximal MCA diameter (SAH=38+10 mm, n=8; sham=88+12 mm, n=7, p<0.01). Basilar artery diameters were similar between sham (165+31 mm, n=7) and SAH (171=15 mm, n=8) animals (p=0.62). Neurologic function was worsened three days after SAH (11 (7-17)) versus wild type shams (27 (27)), p<0.01). SAH grade was 4 (3-4) for SAH mice. No hemorrhage was observed in the sham animals. When both sham and SAH animals were included, proximal MCA diameter correlated with neurological score (p<0.01). Both proximal MCA diameter and neurologic score correlated (p<0.01) with a SAH grade. These methods and procedures can be used to study the effects of ethyl nitrite such as in Background Example 2.

### Background Example 2

8-10 week old male C57B1/6J mice were subjected to either intraluminal filament-induced SAH or sham surgery. Body temperature was controlled at 37° C. during surgery. At 60 min after surgery, mice were assigned to inhale 20 ppm ENO in air or air alone for 3 days in a chamber containing 21% oxygen balanced with nitrogen. Body weight and rotarod performance were examined prior to surgery and 3 days after surgery. Mice were perfused with heparinized saline and formalin. Then the vessels were casted with black ink-gelatin at a pressure of 100 mmHg. Blood clot distribution was assessed. Major arterial diameters (e.g. internal carotid, anterior and middle cerebral and basilar arteries) and cortical tissue relative optical density (ROD) were measured with an image analyzer by an experimenter blind to group assignment. An additional 10 mice were used to measure blood pressure with or without ethyl nitrite (ENO) inhalation.

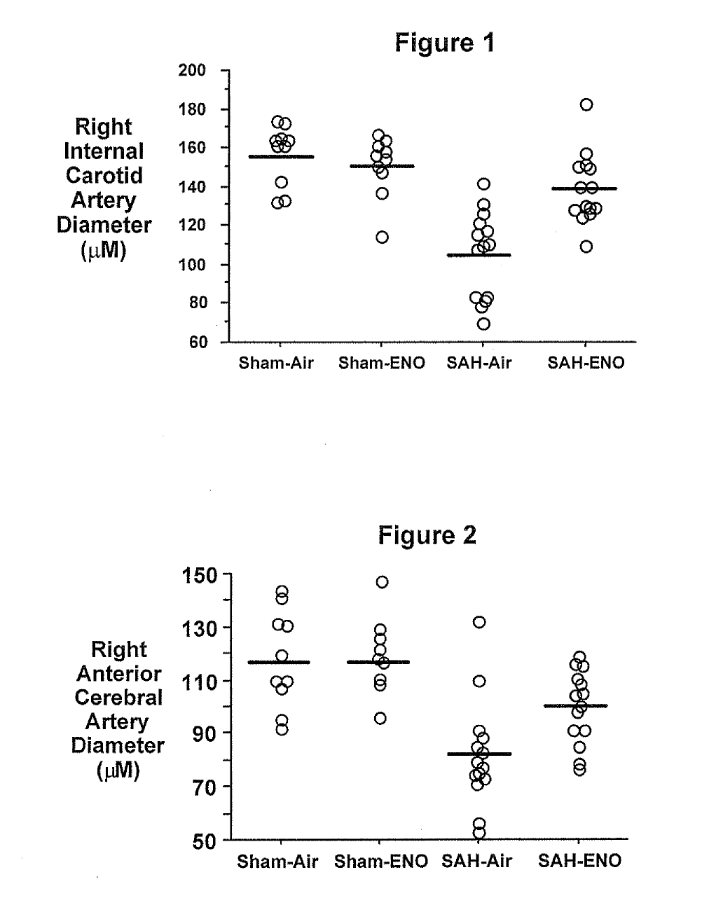

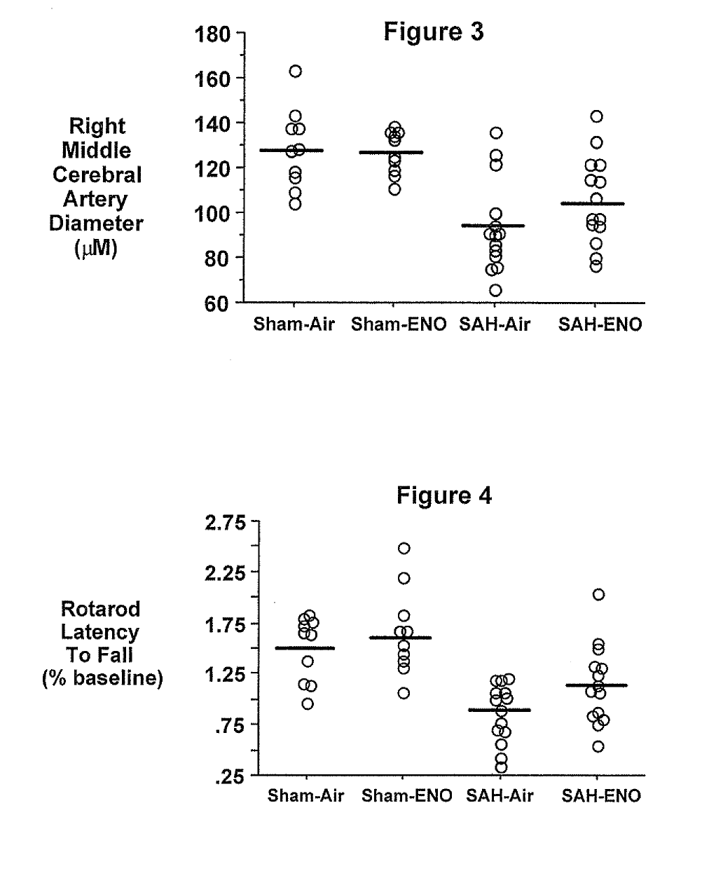

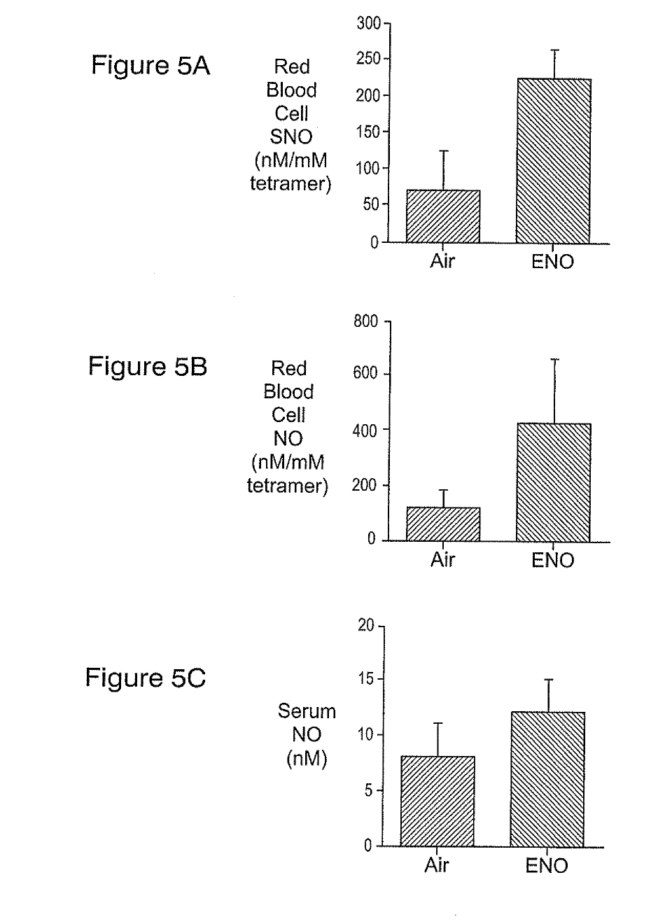

Blood clot distribution was similar in both SAH groups (air n=10, ENO n=14, p=0.8). Arterial narrowing and decreased forebrain ROD were present in SAH mice (p<0.01) and represented large vessel and small vessel spasm, respectively. Both were improved by 20 ppm ENO inhalation (p<0.01, one-way ANOVA, see FIGS. 1-4). There was no effect of ENO in sham surgery mice (n=5 in each group, p=0.9). Inhalation of 20 ppm ENO did not cause a change in blood pressure in normal mice (n=5 each group). Rotarod performance at 3 days post-SAH was worse in SAH mice (p<0.01) (relative to baseline) and a trend for improvement was observed by inhalation of 20 ppm ENO (p=0.06).

Thus, the result was that inhalation of ENO at 20 ppm for 3 days attenuated subarachnoid hemorrhage induced vasospasm without causing systemic hypotension. Vasodilation effect was measured and confirmed 3 days after initiation of treatment as determined by cerebral vessel casting and image analysis of vessel internal diameters.

### Background Example 3

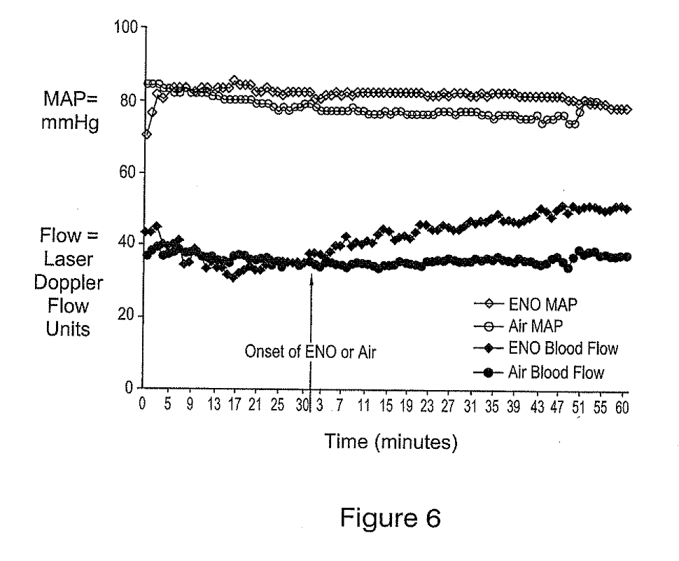

Mice were subjected to subarachnoid hemorrhage induced by filament perforation of the right middle cerebral artery (MCA). Mice were allowed to recover for 7 days. The mice were then anesthetized and a laser Doppler flow probe was positioned over the brain within the territory perfused by the MCA and blood pressure was recorded. The animals were allowed to stabilize for 30 minutes (time=1-30 min). After 30 minutes, the inspiratory gas mixture either remained unchanged (Air) or 20 ppm ENO was added (ENO). Mean arterial pressure remained unchanged under both conditions. Blood flow already reduced below normal values by the subarachnoid hemorrhage, rapidly increased by 25% with the onset of ENO, in contrast to no blood flow change in the control (Air) mice. FIG. 6 shows the results of the experiments.

### Working Example 1

A 70 year old male presents with severe headache, vomiting and dizziness and is diagnosed with subarachnoid hemorrhage by CT scan. The patient within 4 hours after the occurrence of the subarachnoid hemorrhage is given ethyl nitrite at 70 ppm in an admixture of O₂, N₂ and ethyl nitrite such that Paₒ₂ is maintained >90 for three days. Vasospasm does not occur and cerebral infarction is prevented.

### Working Example II

The patient of Working Example I is instead given butyl nitrite dissolved in ethanol intravenously at a dosage of 10 nmol/kg with or without sodium nitrite dissolved in saline at a dosage of 50 nmol/kg. The treatment is started within 4 hours after the occurrence of the subarchnoid hemorrhage. Vasospasm does not occur and cerebral infarction is prevented. Mean arterial blood pressure is not reduced by more than 10%.

### Working Example III

The patent of Working Example I is instead given intravenously sodium nitroprusside (SNP) at a dosage of 1 nmol/kg and potassium nitrite at a dosage of 50 nmol/kg. The dosage of SNP is below that which reduces mean arterial blood pressure by more than 10%. Treatment is started within three hours after the occurrence of subarachnoid hemorrhage. Vasospasm does not occur. Mean arterial blood pressure is not reduced more than 10%.

### Variations

The foregoing description of the invention has been presented describing certain operable and preferred embodiments. It is not intended that the invention should be so limited since variations and modifications thereof will be obvious to those skilled in the art, all of which are within the spirit and scope of the invention.

What is claimed is:

1. A method for attenuating or preventing pathological cerebral vasoconstriction in a patient with subarachnoid hemorrhage, comprising:
administering to the patient a therapeutically effective amount of a compound which mediates an increase of bioactive nitric oxide in blood or tissue in the subarachnoid space to cause vasodilation in cerebral, carotid and basilar arteries after the administration of the compound, and wherein the administration of the compound does not reduce mean arterial blood pressure by more than 15%, and preferably 10%.

2. The method according to claim 1, wherein the compound is administered within 0 to 10 days of the occurrence of the subarachoid hemorrhage.

3. The method according to claim 2, wherein the compound is administered for a period of 1 to 14 days.

4. The method according to claim 1, wherein the compound is an organic nitrite.

5. The method according to claim 4, wherein the compound is administered in the form of a gas.

6. The method according to claim 5, wherein the compound is in a concentration of 1 to 100 ppm.

7. The method according to claim 4, wherein the compound is selected from the group consisting of methyl nitrite, ethyl nitrite, tert-butyl nitrite, and isoamyl nitrite.

8. The method according to claim 6, wherein the compound is ethyl nitrite.

9. The method according to claim 7, wherein the compound is ethyl nitrite.

10. The method according to claim 1, wherein the compound is a nitrosylating agent administered in combination with an inorganic nitrite and/or organic nitrite.

11. The method according to claim 10, wherein the nitrosylating agent is selected from the group consisting of O-nitroso compounds, N-nitroso compounds, S-nitroso compounds, iron nitroso compound, and C-nitroso compounds.

12. The method according to claim 11, wherein the nitrosylating agent is selected from the group consisting of sodium nitroprusside, diethylene triamine NONOate, S-nitrosoglutathione, and S-nitrosopenicilamine.

13. The method according to claim 12, wherein the nitrosylating agent is administered intravenously at a dosage of 1400 nmol/kg.

14. The method according to claim 13, wherein inorganic nitrite is injected or administered intravenously at a dosage of 10 nM to 50 micromolar final plasma concentration.

15. The method according to claim 4, further comprising administering to the patient an inorganic nitrite.

16. The method according to claim 15, wherein the inorganic nitrite is injected or administered intravenously at a dosage of 10 nM to 50 micromolar final plasma concentration.

17. A method for reducing the likelihood or severity of vasospasm in a patient, comprising:
administering to the patient a therapeutically effective amount of a compound which mediates an increase of bioactive nitric oxide in blood or tissue in the subarachnoid space to cause vasodilation in cerebral, carotid and basilar arteries after administration of the compound, and wherein the administration of the compound does not reduce mean arterial blood pressure by more than 10%.

18. The method according to claim 17, wherein the compound is administered within 0 to 10 days after the occurrence of the subarachoid hemorrhage.

19. The method according to claim 18, wherein the compound is administered for a period of 1 to 14 days.

20. The method according to claim 17, wherein the compound is ethyl nitrite and the ethyl nitrite is administered in the form of a gas.

21. The method according to claim 20, wherein the ethyl nitrite is in a concentration of 1 to 100 ppm.

22. The method according to claim 17, wherein the compound is a nitrosylating agent administered in combination with an inorganic nitrite and/or organic nitrite.

23. The method according to claim 22, wherein the nitrosylating agent is selected from the group consisting of O-nitroso compounds, N-nitroso compounds, S-nitroso compounds, iron nitroso compound, and C-nitroso compounds.

24. The method according to claim 23, wherein the nitrosylating agent is selected from the group consisting of sodium nitroprusside, diethylene triamine NONOate, S-nitrosoglutathione, and S-nitrosopenicillamine.

25. The method according to claim 24, wherein the nitrosylating agent is administered intravenously at a dosage of 1-100 nmol/kg.

26. The method according to claim 25, wherein inorganic nitrite is injected or administered intravenously at a dosage of 10 nM to 50 micromolar final plasma concentration.
